SEQUENCE LISTING (3 sequences)

SEQ ID NO: 1 - protein, 13 aa, Artificial Sequence
  Synthetic fibrinogen gamma-chain terminal sequence
CHHLGGAKQAGDV

SEQ ID NO: 2 - protein, 7 aa, Artificial Sequence
  Synthetic oligopeptide
CGGRGDF

SEQ ID NO: 3 - protein, 4 aa, Artificial Sequence
  Synthetic fibronogen alpha 95-98
RGDF

(19) United States
(12) Patent Application Publication
(10) Pub. No.: US 2008/0241233 A1
(43) Pub. Date: Oct. 2, 2008

(54) TARGETED DELIVERY AND EXPRESSION OF PROCOAGULANT HEMOSTATIC ACTIVITY

(75) Inventors: Peter J. Sims, Pittsford, NY (US); Pamela B. Conley, Palo Alto, CA (US); Peng Luan, Redwood City, CA (US); David R. Phillips, San Mateo, CA (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: PORTOLA PHARMACEUTICALS, INC.

(21) Appl. No.: 12/057,100

(22) Filed: Mar. 27, 2008

Related U.S. Application Data
(60) Provisional application No. 60/908,575, filed on Mar. 28, 2007.

(51) Int. Cl. A61K 9/127; A61K 38/02; A61K 39/395; A61K 38/17; A61K 38/43

(52) U.S. Cl. 424/450; 514/2; 424/94.3; 424/130.1; 514/8

(57) ABSTRACT

A platelet substitute consisting of large unilamellar lipid vesicles that contain phosphatidylserine or another procoagulant (clot-promoting) phospholipid, a protein that has binding affinity for collagen or other component of the vessel wall that becomes exposed upon vessel injury, and/or a phospholipid scramblase, has been developed. This platelet substitute provides a means for selectively delivering procoagulant phospholipids and/or fatty acids to the site of vessel injury through targeted adherence to collagen or other component exposed upon vessel injury. These are particularly effective due to the combination of targeting procoagulant vesicles to a site of injury, and triggered exposure of phosphatidylserine (PS) on the surface.

### CLAIM TO PRIORITY

This application claims priority under 35 U.S.C. 119 to U.S. Ser. No. 60/908,575 “Targeted Delivery and Expression of Procoagulant Hemostatic Activity” filed Mar. 28, 2007 by Pamela B. Conley, Peter J. Sims, Peng Luan and David R. Phillips.

### FIELD OF THE INVENTION

The present invention is generally in the field of artificial or substitute platelets.

### BACKGROUND OF THE INVENTION

The present invention is generally in the field of artificial or substitute platelets.

The term “artificial blood” is really a misnomer. The complexity of blood is far too great to allow for absolute duplication in a laboratory. Instead, researchers have focused their efforts on creating artificial substitutes for two important functions of blood: oxygen transport by red blood cells and hemostasis by platelets. As described by Kresie, Artificial blood: An Update on Current Red Cell and Platelet Substitutes Proc (Baylor Univ, Med. Cent.). 2001 April; 14(2): 158-161, a number of driving forces have led to the development of artificial blood substitutes. One major force is the military, which requires a large volume of blood products that can be easily stored and readily shipped to the site of casualties. Another force is the presence in blood or blood cells of potentially infectious agents that introduce the risk of transmitting disease to the transfusion recipient. Examples include HIV and other viruses including Hepatitis C for which diagnostic testing is not adequate to completely eliminate risk, as well as newly-emerging viruses and other known or potential infectious agents such as prion proteins for which no suitable screening procedure to eliminate risk of transfusion borne disease is available. A third force is the growing shortage of blood donors. Approximately 60% of the population is eligible to donate blood, but fewer than 5% are regular blood donors. A unit of blood is transfused every 3 seconds in the USA, and the number of units transfused each year has been increasing at twice the rate of donor collection.

Artificial blood products offer many important benefits. First, they are readily available and have a long shelf life, allowing them to be stocked in emergency rooms and ambulances and easily shipped to areas of need. Second, they can undergo filtration and pasteurization processes to virtually eliminate microbial contamination. No product can claim to be 100% risk-free for infectious agents, but these substitutes have a greatly increased level of safety. Third, they do not require blood typing, so they can be infused immediately and for all patient blood types. Fourth, they do not appear to cause immunosuppression in the recipient.

The greatest progress in the field of blood substitutes has been with the oxygen-carrying solutions. However, research on platelet substitutes has been under way since the 1950s. Current technology/treatment for thrombocytopenia or treatment of hemorrhage accompanying trauma is transfusion of platelets. Risks associated with platelet transfusions are: (1) limited number of platelets harvested from a single donor necessitates transfusion of large number of units that are pooled from multiple donors, increasing the risk for exposure to blood-born viral, bacterial, or prion pathogens (2) risk of leukocyte contamination in platelet preps leading to development of graft versus host disease in the recipient.

One of the biggest factors pushing the need for platelet alternatives is the five-day shelf life of the current blood product. This rapid outdate adds additional constraints to an already limited supply. The platelets are also stored at room temperature, thus increasing the risk of bacterial overgrowth. The risk of bacterial contamination of random donor platelets has been estimated to be 1:1500. Ideally, a platelet substitute would have the following properties: effective hemostasis with a significant duration of action, no associated thrombogenicity, no immunogenicity, sterility, long shelf life with simple storage requirements, and easy preparation and administration.

The advantages of “artificial platelets” over currently available therapies include unlimited production under aseptic conditions, product contains no human-derived material, thus free of potential human pathogens, long-term stability and storage, immunologically inert, targeted selectively to sites of injury (potential for high therapeutic efficacy at low dose and low blood concentration), and non-thrombogenic until activated upon adhesion, thus alleviating the potential danger of DIC (disseminated intravascular coagulation).

Several different forms of platelet substitute are now under development: infusible platelet membranes (IPM), thrombospheres, and lyophilized human platelets.

A lyophilized platelet product has been under development since the late 1950s. The current process involves briefly fixing human platelets in paraformaldehyde prior to freeze-drying in an albumin solution. The fixation step kills microbial organisms, and the freeze-drying greatly increases the shelf life. The adhesive properties of the platelets appear to be maintained.

Infusible platelet membranes are produced from outdated human platelets. The source platelets are fragmented, virally inactivated, and lyophilized. They can then be stored up to two years. Although the platelet membranes still express some blood group and platelet antigens, they appear to be resistant to immune destruction. The product has successfully stopped bleeding in about 60% of such patients. Overall, the product appears to be safe. No adverse effects have been noted, and there is no evidence that those who receive this product have an increased risk of thrombosis.

A liposome based platelet substitute, the plateletsome, with hemostatic efficacy, is described by Rybak, et al., Biomater Artif Cells Immobilization Biotechnol. 1993; 21(2):101-18. A deoxycholate extract of a platelet membrane fraction, with a minimum of 15 proteins including GPIb, GPIIb-IIIa and GPIV/III was incorporated into sphingomyelin: phosphatidylcholine: monosialylganglioside or egg phosphatidylcholine small unilamellar vesicles by reverse-phase/sonication and French press extrusion. These plateletsomes decreased bleeding by 67% in the tail bleeding time in rats made thrombocytopenic (platelets <30,000/microliters) with external irradiation (7-9 Gy) by Cesium source. Efficacy was also demonstrated in the thrombocytopathic, Fawn-Hooded rat, but to a lesser extent than in the thrombocytopenic animals. Direct plateletsome infusion to the tail wound was more effective than systemic administration for all effective preparations. On post-mortem examination, no pathologic thrombi were detected by gross and histopathologic examination of the lungs, livers, kidneys, or spleens of thrombocytopenic or normal animals after plateletsome infusion. No evidence of intravascular coagulation, monitored by levels of circulating fibrinogen and platelet counts, was observed when plateletsomes were administered intravenously to rabbits. No deleterious effect, either inhibition or hyperaggregability, on platelet aggregation studies in vitro was observed.

Thrombospheres (Hemosphere, Irvine, Calif.) are not platelets; but are composed of cross-linked human albumin with human fibrinogen bound to the surface. Experimentally, the thrombospheres appear to enhance platelet aggregation but do not themselves activate platelets. A similar product, Synthocytes (Andaris Group Ltd, Nottingham, UK), has been in clinical trials in Europe, as reported by Davies, et al., Platelets. 2002 June; 13(4): 197-205. Synthocytes are composed of fibrinogen adsorbed on heat stabilized albumin microcapsules of defined size. Synthocytes were found to interact with platelets as shown by platelet aggregation assays and measurements of [¹⁴C]5HT release from platelets in whole blood and platelet-rich plasma. Platelet-Synthocytes co-aggregate formation was demonstrated directly using flow cytometry and the presence of activated platelets in these co-aggregates was demonstrated using an antibody to P-selectin. Synthocytes enhanced platelet responsiveness to conventional aggregating agents such as ADP. Indeed, antagonists of the action of ADP on platelets inhibited the direct effects of Synthocytes on platelets in whole blood, as did a GPIIb/IIIa antagonist. Enhancement of annexin V binding was also observed, indicative of increased pro-coagulant activity. See also Levi, et al. Nat. Med. 1999 January; 5(1):107-11; Nat. Med. 1999 January; 5(1):17-8.

As reported by Teramura, et al. Biochem Biophys Res Commun. 2003 Jun. 20; 306(1):256-60, the recombinant fragment of the platelet membrane glycoprotein Ia/IIa (rGPIa/IIa) has been conjugated to polymerized albumin particles (polyAlb) with an average diameter of 180 nm. The intravenous administration of rGPIa/IIa-polyAlb to thrombocytopenic mice significantly reduced their bleeding time. It was confirmed that rGPIa/IIa-polyAlb had recognition ability against collagen and could contribute to the hemostasis in the thrombocytopenic mice as a platelet substitute.

Takeoka, et al., Biochem Biophys Res Commun. 2003 Dec. 19; 312(3):773-9, describes binding two oligopeptides to latex beads. The oligopeptides were CHHLGGAKQAGDV (SEQ ID NO: 1) (H12), which is a fibrinogen gamma-chain carboxy-terminal sequence (gamma 400-411), and CGGRGDF (SEQ ID NO: 2) (RGD), which contains a fibrinogen alpha-chain sequence (alpha 95-98 RGDF (SEQ ID NO: 3)). Both peptides contained an additional amino-terminal cysteine to enable conjugation. Human serum albumin was adsorbed onto the surface of latex beads (average diameter 1 microm) and pyridyldisulfide groups were chemically introduced into the adsorbed protein. H12 or RGD peptides were then chemically linked to the modified surface protein via disulfide linkages. H12- or RGD-conjugated latex beads prepared in this way enhanced the in vitro thrombus formation of activated platelets on collagen-immobilized plates under flowing thrombocytopenic-imitation blood. Based on the result of flow cytometric analyses of agglutination, PAC-1 binding, antiP-selectin antibody binding, and annexin V binding, the H12-conjugated latex beads showed minimal interaction with non-activated platelets. These results indicate the potential of H12-conjugated particles as a candidate for a platelet substitute.

Severe thrombocytopenia frequently occurs in patients receiving chemotherapy and in patients with autoimmune disorders. Thrombocytopenia is associated with bleeding, which may be serious and life threatening. Current treatment strategies for thrombocytopenia may require transfusion of allogeneic platelets, which is associated with serious drawbacks. These include the occurrence of anti-platelet antibodies, which may result in refractoriness to further platelet transfusions, and the potential risk of transfer of blood-borne diseases. Chemotherapy can also cause deficiencies in platelets. While the above products may be useful in these applications, it is clear that none are effective in all situations.

Therefore, there is a need for a platelet substitute that not only corrects the prolonged bleeding time in individuals rendered thrombocytopenic either by anti-platelet antibodies or by chemotherapy, but also bleeding from surgical wounds or injuries.

### SUMMARY OF THE INVENTION

A platelet substitute consisting of large unilamellar lipid vesicles that contain phosphatidylserine or another procoagulant (clot-promoting) phospholipid, a protein that expresses binding affinity for collagen or other component of the vessel wall that becomes exposed upon vessel injury, and/or a phospholipid scramblase, has been developed. This platelet substitute provides a means for selectively delivering procoagulant phospholipids and/or fatty acids to the site of vessel injury through targeted adherence to collagen or other component(s) exposed upon vessel injury. These are particularly effective due to the combination of targeting procoagulant vesicles to a site of injury, and triggered exposure of phosphatidylserine (PS) on the surface.

In one embodiment, a protein targeting to and adhering to subendothelium is bound to the outer surface of lipid vesicles that are prepared with phospholipids (PL) that are randomly distributed in the membrane, with an amount of phosphatidylserine (PS) that is sufficient to provide procoagulant function, and the remaining PL are chosen from lipids such as phosphatidylcholine (PC), sphingomyelin (SM), and phosphatidylethanolamine (PE). In another embodiment, asymmetric lipid vesicles are prepared selectively incorporating a procoagulant phospholipid such as PS within the inner leaflet of the vesicle membrane, wherein the outer leaflet of the vesicle membrane consists of PC or another neutral phospholipid such as SM. In this embodiment (asymmetric lipid vesicle) both a protein targeting to and adhering to subendothelium and a phospholipid scramblase protein are incorporated into the membrane of the lipid vesicle.

The targeting protein and the phospholipid scramblase can be incorporated into the membrane of the lipid vesicles together as a recombinant chimeric protein or separately. In a preferred embodiment, a single chimeric protein construct consisting of the protein targeting to and adhering to the subendothelium covalently linked through a transmembrane amphipathic helix to a phospholipid scramblase is incorporated into the membrane of the lipid vesicle, with the subendothelium-targeting domain of the chimeric protein exposed on the external surface of the lipid vesicle and the phospholipid scramblase domain of the chimeric protein located on the internal surface of the lipid vesicle. In this embodiment, the PL of the vesicle membrane is asymmetrically distributed, with PS concentrated in the inner leaflet of the vesicle membrane and PC or SM concentrated in the outer leaflet of the vesicle membrane. In the preferred embodiment, the proteins are provided as a recombinant chimeric protein consisting of a targeting protein such as the extracellular domain of human platelet glycoprotein GPIa-IIa, which is covalently linked to phospholipid scramblase 1 (PLSCR1) through a transmembrane domain that is capable of facilitating leakage of calcium ion across the vesicle membrane. In another embodiment, the targeting function is provided by another collagen receptor, by a receptor for another component of the subendothelium (e.g., GPIb for vWf), or by antibody specific for collagen or another subendothelial component. In another embodiment, human phospholipid scramblase 1 (PLSCR1) is replaced by another protein of the phospholipid scramblase gene family, such as human PLSCR2, PLSCR3, or PLSCR4 (Wiedmer, et al. Biochim Biophys Acta. 2000 Jul. 31; 1467(1):244-53.). In another embodiment, the targeting protein is covalently coupled directly to the phospholipid head group of PE and the PE contains lipid-bilayer-perturbing fatty acid chains attached at the SN1 or SN2 positions of the glycerol backbone of the phospholipid.

The platelet substitutes can be prepared and stored at 4° C., frozen at −20° C., or, as lyophilized or dried preparations, which are reconstituted in sterile saline or a buffered solution before administration by intravenous injection or topical administration directly at the site of a wound or during surgery, as a hemostatic.

### BRIEF DESCRIPTION OF THE DRAWINGS

FIG. 1 is a graph of the specific binding (measured as fluorescence) of collagen-antibody-conjugated vesicles to collagen surface as a function of nmoles of lipid. Collagen-antibody-conjugated vesicles bind to collagen surface in a dose-dependent manner (col-Ab vesicle/col), but not to BSA coated surface (col-AB vesicle/BSA). The binding of un-conjugated control vesicles to collagen surface under the same conditions is shown as unconjugated vesicle/col. Vesicles contain 20% porcine brain PS, 1% NBD-PC, 3% PEG-2000-DSPE (1,2-Distearoyl-sn-Glycero-3-Phospoethanolamine-N-[Methoxy(Polyethylene glycol)-2000]), 1% PEG-2000-DSPE-MAL, and 75% egg PC.

FIG. 2 is a graph of the procoagulant activity (measured as thrombin activity, Vmax) of vesicles after conjugation of anti-collagen antibody on PS-containing vesicle (as a function of micromolar lipid in assay), showing that conjugation does not affect the procoagulant activity of the vesicles. 4 vesicle preparations, collagen-antibody-conjugated vesicles with 20% phosphatidylserine (PS, col-AB), control-antibody-conjugated vesicles with 20% PS (control AB), un-conjugated vesicles with 20% PS (unconjugated) and un-conjugated phosphatidylcholine (PC) vesicles (PC vesicle) were tested in thrombin generation assay. Vesicles contain 20% porcine brain PS, 1% NBD-PC, 3% PEG-2000-DSPE (1,2-Distearoyl-sn-Glycero-3-Phospoethanolamine-N-[Methoxy(Polyethylene glycol)-2000]), 1% PEG-2000-DSPE-MAL, and 75% egg PC. In PC vesicle, 20% PS is replaced with PC. In this experiment, there are 31 antibodies per vesicle on average for antibody conjugated vesicles.

FIG. 3 is a graph of the prothrombinase activity on PS-containing vesicles conjugated with collagen antibody, showing they are procoagulant when they are specifically targeted to collagen surface. Vesicles are allowed to bind to collagen (or BSA) coated plate. After the unbound vesicles are washed away, thrombin generation assay is performed for each sample. The graph shows thrombin activity generated in each sample. Shown in the graph from left to right are: un-conjugated PC vesicles on collagen; un-conjugated PS vesicles on collagen; non-specific-antibody-conjugated PS vesicles on collagen; collagen-antibody-conjugated PS vesicles on collagen and collagen-antibody-conjugated PS vesicles on BSA.

FIG. 4 is a graph of the specific binding of collagen-antibody-conjugated vesicles to collagen-coated glass capillary under flow conditions. Binding of each vesicle preparation is expressed as the mean fluorescence of a capture field, n=4, Vesicles contain 20% porcine brain PS, 1% NBD-PC, 3% PEG-2000-DSPE (1,2-Distearoyl-sn-Glycero-3-Phospoethanolamine-N-[Methoxy(Polyethylene glycol)-2000]), 1% PEG-2000-DSPE-MAL, and 75% egg PC.

FIG. 5 is a graph of formation of fibrin by collagen-antibody-conjugated vesicles on collagen surface in whole blood when platelets are inhibited by 318, an inhibitor of platelet adhesion and activation. The capillaries are coated with collagen (collagen:tissue factor ratio is 500:1). The positive control is whole blood with Integrilin (a GPIIbIIIa antagonist). Fibrin formation was measured when 318 was added to whole blood plus Integrilin. A second control measured fibrin formed by control vesicles conjugated with a non-specific antibody in the presence of 318. Blood was collected rapidly in 2.4 μm (final) Integrilin, and perfused through the collagen:tissue factor coated capillary at a shear rate of 300 s⁻¹.

### DETAILED DESCRIPTION OF THE INVENTION

### I. Definitions

As used herein, procoagulant (clot-promoting) phospholipids refer to those lipids, such as 1-phosphatidylserine, that accelerate the generation of thrombin from its proenzyme, prothrombin when the lipid is introduced to blood or plasma.

As used herein, neutral lipid refers to phosphatidylcholine, sphingomyelin, or other lipid that does not carry a net negative electronic charge.

As used herein, phospholipid scramblase refers to Phospholipid scramblase 1 (PLSCR1) an interferon (IFN)- and growth factor-inducible, calcium-binding protein that either inserts into the plasma membrane or binds DNA in the nucleus depending on its state of palmitoylation. In certain hematopoietic cells, PLSCR1 is required for normal maturation and terminal differentiation from progenitor cells as regulated by select growth factors, where it promotes recruitment and activation of Src kinases. PLSCR1 is a substrate of Src (and Abl) kinases, and transcription of the PLSCR1 gene is regulated by the same growth factor receptor pathways in which PLSCR1 potentiates afferent signaling. Results suggest that PLSCR1, which is itself an ISG-encoded protein, provides a mechanism for amplifying and enhancing the IFN response through increased expression of a select subset of potent antiviral genes (Dong, et al. J. Virol. 2004 September; 78(17):8983-93).

As used herein, subendothelium refers to the connective tissue between the endothelium and the inner elastic membrane in the intima of arteries. Examples of components which are exposed exclusively or preferentially at the time of injury or exposure of the subendothelium include collagen, von Willebrand factor, and laminin.

As used herein, the term “antibodies” include any antigen binding molecule, including recombinant, single chain, enzyme fragments, polyclonal and monoclonal antibodies, unless otherwise specified.

As used herein, lipid-bilayer-perturbing fatty acid chains refer to long fatty acid chains that perturb the lipid bilayer. Some examples are C14-C18 saturated (i.e., no double bonds) and monosaturated (i.e., one double bond) fatty acids. Unsaturated fatty acids increase membrane fluidity. Double bonds in the fatty acid chain in the cis configuration cause the chain to bend. Saturated fatty acids are also disruptive. This may be due simply to the length/flexibility of the hydrophobic tail.

Phospholipids are non-immunogenic natural or synthetic molecules including a glycerol backbone having acyl chains including a neutral or charged headgroup covalently bound to the glycerol backbone at SN1 and SN2 positions.

A procoagulant amount can be measured using any of the in vitro or in vivo assays for prothrombin conversion to thrombin. Representative in vitro assays including the one-stage clotting assay, the thrombinase assay (measuring thrombin production) in the examples and the de-dimerize assay (measuring fibrin generation). A shortening of the in vivo bleeding time can also be used.

As used herein, a chimera or chimeric protein is a human-engineered protein that is encoded by a nucleotide sequence made by splicing together of two or more complete or partial genes that encode a chimeric protein.

### II. Platelet Compositions

The platelet substitutes include:

large unilamellar lipid vesicles that contain a procoagulant amount of phosphatidylserine or another procoagulant (clot-promoting) phospholipid such as phosphatidylcholine (PC), sphingomyelin (SM), and phosphatidylethanolamine (PE), and

a protein that expresses binding affinity for collagen or other component of the vessel wall that becomes exposed upon vessel injury and/or a phospholipid scramblase.

In one embodiment, the protein targeting to and adhering to subendothelium is covalently bound to the outer surface of lipid vesicles that are prepared with phospholipids (PL) that are randomly distributed in the membrane. For example, the targeting protein is covalently coupled directly to the phospholipid head group of PE and the PE contains lipid-bilayer-perturbing fatty acid chains attached at the SN1 or SN2 positions of the glycerol backbone of the phospholipid. In another embodiment, asymmetric lipid vesicles are prepared selectively incorporating a procoagulant phospholipid such as PS within the inner leaflet of the vesicle membrane, wherein the outer leaflet of the vesicle membrane consists of PC or another neutral phospholipid such as SM. In this embodiment (asymmetric lipid vesicle), a protein targeting to and adhering to subendothelium and, optionally, a phospholipid scramblase protein, are incorporated into the membrane of the lipid vesicle. The targeting protein and the scramblase are incorporated in an amount of at least one molecule each per vesicle up to ½ of the number of lipid molecules.

The targeting protein and the phospholipid scramblase can be incorporated into the membrane of the lipid vesicles together as a recombinant chimeric protein or separately. In a preferred embodiment, a single chimeric protein construct consisting of the protein targeting to and adhering to the subendothelium covalently linked through a transmembrane amphipathic helix to a phospholipid scramblase is incorporated into the membrane of the lipid vesicle, with the subendothelium-targeting domain of the chimeric protein exposed on the external surface of the lipid vesicle and the phospholipid scramblase domain of the chimeric protein located on the internal surface of the lipid vesicle. In this embodiment, the PL of the vesicle membrane is asymmetrically distributed, with PS concentrated in the inner leaflet of the vesicle membrane and PC or SM concentrated in the outer leaflet of the vesicle membrane. In the preferred embodiment, the proteins are provided as a recombinant chimeric protein consisting of a targeting protein such as the extracellular domain of human platelet glycoprotein GPIa-IIa, which is covalently linked to phospholipid scramblase 1 (PLSCR1) through a transmembrane domain that is capable of facilitating leakage of calcium ion across the vesicle membrane.

A. Large Unilamellar Lipid Vesicles

As used herein, large unilamellar lipid vesicles, “LUV”, can be prepared by a variety of methods including extrusion (LUVET or “Large, Unilamellar Vesicles prepared by Extrusion Technique”), detergent dialysis (DOV or Dialyzed Octylglucoside Vesicles), fusion of SUV (FUV or “Fused, Unilamellar Vesicles”), reverse evaporation (REV or “Reverse Evaporation Vesicles), and ethanol injection. Unilamellar vesicles are prepared from MLV or LMV (Large, Multilamellar Vesicles), the large “onion-like” structures formed when amphiphilic lipids are hydrated. Small, unilamellar vesicles, “SUV”, are small, unilamellar vesicles” and are usually prepared by sonication using a cuphorn, bath, or probe tip sonicator. SUV are typically 15-30 nm in diameter while LUV range from 100-200 nm or larger. LUV are stable on storage, however, SUV will spontaneously fuse when they drop below the phase transition temperature of the lipid forming the vesicle. Lipids used in the preparation of the liposomes can include cholesterol, phosphatidylserine, phosphatidylethanolamine, phosphatidylcholine, phosphatidylglycerol, and/or sphingomyelin, as well as the PEGylated or N-glutaryl derivatives thereof. The carbonyl acyl chain tail groups attached at the SN-1 and SN-2 positions of the phospholipids can range from C:12-28, with varying degrees of bond saturation, the chain length, chain saturation, and molar ratios of the incorporated lipids each are selected so as to optimize liposome size, fluidity, stability, and pharmacologic activity. Sucrose or another disacharide cryoprotectant may be added to enhance preservation under conditions of lyophilization.

In the preferred embodiment, the protein molecules and clot-promoting phospholipids are attached to or incorporated into large unilamellar lipid vesicles. In the most preferred embodiment, the lipid vesicles are prepared having a procoagulant phospholipid such as 1-phosphatidylserine, incorporated within the inner leaflet of the membrane while the outer leaflet is comprised of phosphatidylcholine or another neutral phospholipid such as sphingomyelin. The PS is provided in an amount sufficient to make the vesicles procoagulant, typically 10-90 mol percent of the lipid.

The inner phospholipid is preferably a pro-coagulant phospholipid. Alternatively or in addition, lipid-bilayer-perturbing fatty acid chains can be covalently incorporated into the lipid. Representative fatty acids are C14-C18 saturated (i.e., no double bonds) and monosaturated (i.e., one double bond) fatty acids. Unsaturated fatty acids increase membrane fluidity. Double bonds in the fatty acid chain in the cis configuration cause the chain to bend. Saturated fatty acids are also disruptive. This may be due simply to the length/flexibility of the hydrophobic tail.

Lipid vesicles can be prepared with phospholipids (PL) that are randomly distributed in the membrane, with an amount of phosphatidylserine (PS) that is sufficient to provide procoagulant function, with the remaining PL chosen from among phosphatidylcholine (PC), sphingomyelin (SM), and phosphatidylethanolamine (PE). In another embodiment, asymmetric lipid vesicles are prepared selectively incorporating a procoagulant phospholipid such as PS within the inner leaflet of the vesicle membrane, wherein the outer leaflet of the vesicle membrane consists of PC or another neutral phospholipid such as SM.

In one embodiment, lipid vesicles are prepared with phospholipids (PL) that are randomly distributed in the membrane, with an amount of phosphatidylserine (PS) that is sufficient to provide procoagulant function, and the remaining PL are chosen from among phosphatidylcholine (PC), sphingomyelin (SM), and phosphatidylethanolamine (PE). In another embodiment, asymmetric lipid vesicles are prepared selectively incorporating a procoagulant phospholipid such as PS within the inner leaflet of the vesicle membrane, wherein the outer leaflet of the vesicle membrane consists of PC or another neutral phospholipid such as SM. The phospholipids may be modified, for example, by pegylation. Examples include DSPE-PEG2000, or phosphotidylethanolamine-N-[Methoxy(polyethylene glycol)-2000)).

B. Phospholipid Scramblase

In one embodiment, the platelet substitute contains a phospholipid scramblase. In the embodiment discussed above, the PL of the vesicle membrane are randomly distributed such that PS is always exposed on the outer surface in a quantity that is sufficient to provide procoagulant function and a subendothelial targeting moiety is bound to the outer surface of the vesicle membrane. In this embodiment, there is no phospholipid scramblase component in the vesicle membrane.

The phospholipids (PL) in the plasma membrane of all eukaryotic cells including blood platelets are normally asymmetrically distributed: the inner (endofacial) leaflet is highly-enriched in the aminophospholipids including phosphatidylserine (PS) and phosphatidylethanolamine (PE), whereas the outer (exofacial) leaflet is enriched in the neutral PL, including phosphatidylcholine (PC) and sphingomyelin (SM) (Zwaal et al. Cellular & Molecular Life Sciences. 2005 May. 62(9):971-88). This asymmetric distribution is believed to be established by vectorial PL transporters that use the energy of ATP to concentrate PS and PE in the inner leaflet, with PC and SM concentrated in the outer leaflet. Such ATP-dependent lipid transporters include the aminophospholipid translocase, a P-type ATPase that transports PS and PE from outer to inner plasma membrane leaflet, sequestering these phospholipids to the endofacial (inner) membrane surface. (Daleke. J. Biol. Chem. 2007 Jan. 12, 282(2):821-5.) When platelets are injured or activated, there is a collapse of this normal asymmetric distribution of plasma membrane PL that results in the exposure of PS on the plasma membrane exofacial (outer) surface. This collapse of plasma membrane PL asymmetry under conditions of platelet activation or injury has been shown to result as the consequence of a rise in cytosolic Ca²⁺, which triggers a rapid bidirectional-movement of PL between plasma membrane leaflets with net exposure of PS on the exofacial (outer) surface. De novo exposure of PS on the surface of activated platelets in response to increased intracellular Ca²⁺ is thought to play a key role in expression of platelet procoagulant activity and in clearance of injured or apoptotic cells. (Wolfs et al. Cellular & Molecular Life Sciences. 2005 July, 62(13):1514-25.) This intracellular Ca²⁺-activated “scrambling” of plasma membrane PL that results in expression of platelet procoagulant activity has been attributed to a Ca²⁺-binding endofacial plasma membrane protein designated “phospholipid scramblase” (PLSCR). An approximately 37-kDa protein in erythrocyte membrane that mediates Ca²⁺-dependent movement of PL between membrane leaflets, similar to that observed upon elevation of Ca²⁺ in the cytosol (Basse, et al. J. Biol. Chem. 271, 17205-17210), was isolated.

A 1,445-base pair cDNA was cloned from a K-562 cDNA library (Zhou, et al., J Biol. Chem. (1997) 18; 272(29):18240-4). The deduced “PL scramblase” protein is a proline-rich, type II plasma membrane protein with a single transmembrane segment near the C terminus. Antibody against the deduced C-terminal peptide was found to precipitate the approximately 37-kDa red blood cell protein and absorb PL scramblase activity, confirming the identity of the cloned cDNA to erythrocyte PL scramblase. Ca²⁺-dependent PL scramblase activity was also demonstrated in recombinant protein expressed from plasmid containing the cDNA. Quantitative immunoblotting revealed an approximately 10-fold higher abundance of PL scramblase in platelet (approximately 10⁴ molecules/cell) than in erythrocyte (approximately 10³ molecules/cell), consistent with apparent increased PL scramblase activity of the platelet plasma membrane. PL scramblase mRNA was found in a variety of hematologic and nonhematologic cells and tissues, suggesting that this protein functions in all cells.

Phospholipid scramblase activity and the gene encoding the enzyme is described by Basse, et al. J Biol. Chem. 1996 Jul. 19; 271(29):17205-10; Scott, et al. J Clin Invest. 1997 May 1; 99(9):2232-8. Zhou, et al. J Biol Chem. 1997 Jul. 18; 272(29):18240-4. Zhou, et al., Biochemistry. 1998 Feb. 24; 37(8):2356-60, Zhao, et al. J Biol Chem. 1998 Mar. 20; 273(12):6603-6, and Zhao, et al. Biochemistry. 1998 May 5; 37(18):6361-6.

Elevation of cytosolic Ca²⁺ serves to activate plasma membrane phospholipid scramblase activity and to inhibit the activity of aminophospholipid translocase. The net effect is elevation of PS exposure in those platelets exposed to increased intracellular Ca²⁺. Platelet procoagulant activity is mainly determined by the extent of surface-exposed PS, resulting from its movement from inner to outer plasma membrane leaflet under this condition of activated phospholipid scramblase. Besides the formation of procoagulant microparticles, the results show that a distinct fraction of the platelets exposes PS when stimulated. The extent of PS exposure in these platelet fractions is similar to that in platelets challenged with Ca²⁺-ionophore, where all cells exhibit maximal attainable PS exposure. The size of the PS-exposing fraction depends on the agonist and is proportional to the platelet procoagulant activity. Phospholipid scramblase activity is observed only in the PS-exposing platelet fraction, whereas aminophospholipid translocase activity is exclusively detectable in the fraction that does not expose PS. Procoagulant platelets exhibit maximal surface exposure of PS, the consequence of intracellular Ca²⁺ switching on the activity of phospholipid scramblase and inhibiting the activity of aminophospholipid translocase. (Wolfs et al. Cellular & Molecular Life Sciences. 2005 July, 62(13):1514-25.)

In the preferred embodiment, a chimeric protein construct representing amino acid residues 1-318 of human PLSCR1 (phospholipid scramblase 1) covalently linked by peptide bond of its carboxyl-terminus (residue 318) to a subendothelial targeting moiety is incorporated into the membrane of lipid vesicles. Other embodiments of the phospholipid scramblase domain of the chimeric protein include other members of the phospholipid scramblase family of proteins (PLSCR2, PLSCR3, PLSCR4), or, N-terminal deleted forms of these proteins in which the amino terminal segments of the phospholipid scramblase polypeptide is deleted from residue 1-residue 118. (Wiedmer, et al. Biochim Biophys Acta. 2000 Jul. 31; 1467(1):244-53.). In this preferred embodiment, the PL component of the lipid vesicle membranes are prepared so as to mimic that of the normal platelet plasma membrane in the resting state, where PS is sequestered to the inner leaflet and PC is distributed in the outer leaflet.

C. Targeting Proteins

The vesicles are targeted to components of the subendothelium that are expressed exclusively or preferentially upon exposure of the subendothelium, for example, when the endothelium is injured. An example is collagen. Proteins that selectively bind to collagen include glycoprotein VI, the glycoprotein Ia-IIa complex, glycoprotein Ib, von Willebrand's factor (“vWf”), as well as an antibodies directed against collagen. All of these are commercially available as well as described in the literature. The extracellular domain of human GPIa-IIa is described by Takada et al J Cell Biol. 1989; 109:397-407; Argraves et al J Cell. Biol. 1987; 105:1183-1190. Alternatively, one could use the extracellular domain of human GPIb or a monoclonal or recombinant antibody (Fab, Fab′2 or other antigen-binding fragment) directed against collagen type I, II, III or IV or to human vWF. Also, recombinant C1qTNF-related protein-1 (CTRP-1) has been previously shown to bind fibrillar collagen and block collagen-induced platelet aggregation, and prevented vWF binding to collagen (Lasser et al, Blood 2006: 107:423-430). Thus, this recombinant protein could also be used to target vesicles to a collagen surface.

Previous work has demonstrated that enhanced platelet aggregation can be provided by either latex beads (Okamura et al 2006) or phospholipid vesicles (Okamura et al, 2005, Bioconjugate Chem 16, 1589-1596) that bear a peptide containing a sequence of the fibrinogen gamma-chain carboxy-terminal sequence. Vesicles (220 nm) bearing the fibrinogen dodecapeptide sequence have been demonstrated to enhance the in vitro thrombus formation of platelets that were adhering to a collagen surface, and to decrease the bleeding time of thrombocytopenic rats (Okamura et al, Bioconjugate Chem 2005, 16, 1589-1596). Furthermore, this same group subsequently demonstrated that a mixture of latex beads conjugated either to the fibrinogen dodecapeptide sequence or a recombinant soluble moiety of GPIba was able to enhance platelet thrombus formation in vitro under high shear rates (Okamura et al J Artif. Organs 2006, 9: 251-258).

A critical role of platelets in hemostasis is to provide a charged membrane surface which affords a site of assembly for the procoagulant complexes of Factor IXa/VIIIa and Factor Va/Xa, which generate two important coagulation enzymes in the coagulation cascade, Factor Xa and Thrombin, respectively. The exposure of PS on the platelet surface following platelet activation is critical to this process. Prior art has demonstrated that phospholipid vesicles containing a mixture of phosphotidylcholine: phosphatidylserine (PCPS) can mimic the PS-containing platelet outer membrane and serve as a source of coagulant phospholipid for generation of fXa and thrombin. Previous work by Giles et al (Giles et al, Br. J. Hematology 1988, 69: 491-497) have demonstrated that co-administration of fXa and PCPS vesicles could substitute for fVIII deficiency in hemophilic dogs, in that bleeding time could be shortened, indicating that PCPS vesicles can provide procoagulant activity in vivo.

In one embodiment, a protein targeting to and adhering to subendothelium is bound to the outer surface of lipid vesicles. In another embodiment, asymmetric lipid vesicles are prepared selectively incorporating a procoagulant phospholipid such as PS within the inner leaflet of the vesicle membrane, wherein the outer leaflet of the vesicle membrane consists of PC or another neutral phospholipid such as SM. In this embodiment (asymmetric lipid vesicle) both a protein targeting to and adhering to subendothelium and a phospholipid scramblase protein are incorporated into the membrane of the lipid vesicle.

D. Chimeric Targeting Protein/Phospholipid Scramblase

The targeting protein and the phospholipid scramblase can be incorporated into the membrane of the lipid vesicles together as a recombinant chimeric protein or separately. In the preferred embodiment, a single chimeric protein construct consisting of the protein targeting to and adhering to the subendothelium covalently linked through a transmembrane amphipathic helix to a phospholipid scramblase is incorporated into the membrane of the lipid vesicle, with the subendothelium-targeting domain of the chimeric protein exposed on the external surface of the lipid vesicle and the phospholipid scramblase domain of the chimeric protein located on the internal surface of the lipid vesicle. In this preferred embodiment, the PL of the vesicle membrane is asymmetrically distributed, with PS concentrated in the inner leaflet of the vesicle membrane and PC or SM concentrated in the outer leaflet of the vesicle membrane.

In the preferred embodiment, the proteins are provided as a recombinant chimeric protein consisting of a targeting protein such as the extracellular domain of human platelet glycoprotein GPIa-IIa, which is covalently linked to phospholipid scramblase 1 (PLSCR1) through a transmembrane domain that is capable of facilitating leakage of calcium ion across the vesicle membrane. In another embodiment, the targeting function is provided by another collagen receptor, by a receptor for another component of the subendothelium (e.g., GPIb for vWf), or by antibody specific for collagen or another subendothelial component. In another embodiment, human phospholipid scramblase 1 (PLSCR1) is replaced by another protein of the phospholipid scramblase gene family, such as human PLSCR2, PLSCR3, or PLSCR4 (Wiedmer, et al. Biochim Biophys Acta. 2000 Jul. 31; 1467(1):244-53.). In another embodiment the targeting protein is covalently coupled directly to the phospholipid head group of PE and the PE contains lipid-bilayer-perturbing fatty acid chains attached at the SN1 or SN2 positions of the glycerol backbone of the phospholipid.

### II. Methods of Manufacture

A. Production of LUV

Methods for making LUV are well known. In the preferred embodiment, the subendothelial targeting and scramblase (PLSCR) protein construct are incorporated by detergent dialysis into large unilamellar vesicles (100-200 nm diameter) with bulk lipids composed of synthetic 1-phosphatidylserine (PS), 1-phosphatidylethanolamine (PE), 1-phosphatidylcholine (PC), sphingomyelin (SM), and cholesterol (or other sterol). Addition of cholesterol or other sterol to the lipids comprising such vesicles has been shown to improve stability and reduce leakage of small ions or other solute across the vesicle membrane (see for example, Szoka, Jr. et al. Ann. Rev. Biophys. Bioeng. 9:467-508 (June 1980). After size-selection through polycarbonate filter extrusion, more than 95% of the PS and PE in the outer leaflet of these LUV is then depleted by incubation with non-specific phospholipid exchange protein (PLEP) in the presence of sonicated small unilamellar vesicles comprised exclusively of PC and SM (SUV), the PC/SM SUV present at 20-fold molar phospholipid excess to that of LUV phospholipids, as described by J. A. F. Op den Kamp. Lipid Asymmetry in Membranes. Ann. Rev. Biochem. 1979. 48:47-71, and references therein. Following this PLEP-catalyzed exchange of the outer leaflet lipid, the LUV are separated from the SUV and PLEP by size exclusion chromatography in the presence of sterile, pyrogen-free isotonic saline, and the LUV stored at −20° C. and thawed immediately prior to use. Short term storage at 4° C. is also suitable. Alternatively, the preparation can be lyophilized or dried in a sterile dosage unit container.

B. Targeting and Procoagulant Proteins

Targeting and procoagulant proteins can be mixed with the LUV or provided as a chimeric recombinant protein.

Recombinant protein used for reconstitution of the subendothelial targeting phospholipid scramblase functions of platelets are synthesized from cDNA/plasmid constructs in a bacterial host such as E. coli using standard methods of molecular biology. The preferred construct encodes for a chimeric protein linking human PLSCR1 to the extracellular domain of human GPIa-IIa, with C-terminal (residue 318) of PLSCR1 linked to the extracellular domain of human GPIa-IIa (see Takada et al J Cell Biol. 1989; 109:397-407; Argraves et al J Cell. Biol. 1987; 105:1183-1190) by modification of the methods of Nishiya et al (Blood. 2002; 100:136-142). Alternatively, the chimeric structure can consist of the C-terminus of PLSCR1 coupled to the extracellular domain of human GPIb or to a monoclonal antibody (Fab, Fab′2 or other antigen-binding fragment) directed against type I, II, III or IV human collagen or to human vWF.

C. Assembly of a Platelet Substitute.

Phospholipid vesicles are prepared by dissolving lipids in a solvent such as chloroform, then drying, and hydrated with buffer or other aqueous solution. This is vortexed to resuspend the mixture. Freeze-thaw cycles are used to form vesicles, which are then extruded through a filter such as a 200 micron pore polycarbonate membrane.

### III. Methods of Administration

The platelet substitutes can be prepared and stored refrigerated (4° C.) or as lyophilized or dried preparations, which are reconstituted in sterile saline or a buffered solution before administration by intravenous injection or topical administration directly at the site of a wound or during surgery, as a hemostatic. Dosage will be determined by effective hemostasis. This product may be used in clinical settings where platelet transfusions are normally required, and may include the following examples: patients with thrombocytopenia (due to chemotherapy, myelodysplastic syndrome, or immune thrombocytopenia purpura) or leukemia or undergoing hematopoetic stem cell transplantation, patients undergoing cardiopulmonary bypass surgery or other surgical procedures, patients with platelet dysfunction or patients with acute blood loss (trauma).

The present invention will be further understood by reference to the following non-limiting examples.

### Materials and Methods

### Preparation of Phospholipid Vesicles:

All lipids are purchased from Avanti Polar Lipids. 10 μmoles of lipid mixture in chloroform are dispensed into a flat-bottom glass tube and the mixture is dried under a stream of nitrogen. Residual organic solvent is removed under vacuum for overnight. The dried lipid film is hydrated with 1 ml of buffer V (50 mM Hepes, pH 8.5, 100 mM NaCl and 2.5 mM EDTA) for 1 hour with frequent agitation. The mixture is vortexed for 30 seconds to ensure the complete suspension of lipid residues. The lipid solution is then subjected to 5 cycles of freeze-thaw process (ethanol dry ice and 37° C. water bath). The final lipid vesicle preparation is then obtained by passing the mixture through two layers of polycarbonate filter with 200 nm pores for 15 times, in a Mini-Extruder (Liposofast™, Avestin, Inc., Ottawa, Ontario, Canada). Lipid vesicles intended for conjugation are always used within 30 minutes after preparation. Vesicles contain 1% fluorescently labeled lipid 1-Oleoyl-2-[6-[(7-nitro-2-1,3-benzoxadiazol-4-yl)caproy]-sn-lycero-3-phosphocholine (18:1-06:0 NBD-PC) for visualization and quantification purposes.

### Conjugation of Antibody to Vesicles:

Antibodies purchased from vendors (Rockland, US Biological) are dialyzed against buffer V with two changes of 500 ml overnight at 4° C. For thiolation of antibody, 40-fold (mol/mol) of freshly prepared Traut's reagent (2-iminothiolane, from Pierce) is added to antibody and the reaction is allowed to proceed at room temperature for 1 hour. Excess of Traut's reagent is removed by a size-exclusion column (Pierce Zeta Desalt Spin Column). The thiolated antibody is immediately mixed with freshly made lipid vesicle containing maleimide (1,2-Distearoyl-sn-Glycero-3-Phosphoethenolamine-N-[Maleimide(Polyethylene Glycol)2000] (Ammonium salt), or DSPE-PEG2000-MAL, Avanti Polar Lipids). The conjugation is carried out at room temperature for at least 6 hours.

Purification of Immunoliposome from Free Antibody:

Free antibody in the conjugation mixture is removed from vesicles using sucrose gradient flotation. 0.7 ml vesicle after conjugation is mixed with equal volume of 80% sucrose on the bottom of centrifuge tube. 7 ml of 20% sucrose is carefully placed on top of the sample in tube. Finally 2 ml of 0% sucrose is layered on top. Sucrose solution is made in buffer V minus EDTA. The gradient is centrifuged at 100,000×g for 20 hours in a swinging bucket rotor. Vesicles are collected at the 0/20% sucrose interface. Free un-conjugated antibody is recovered in the 40% sucrose fraction for quantification purpose.

### Quantification of Antibody on Vesicles:

Equal portions of vesicles before and after sucrose purification, as well as free unconjugated antibody, are loaded onto SDS-PAGE for quantification purpose. Antibody in each fraction is quantified using the density of protein band after electrophoresis and staining of the gel. Different amounts of pure antibody are also included on the same gel as standards to ensure that the protein band density is within linear range.

### Binding of Immunoliposome on Collagen Surface Under Static Condition:

Collagen is coated in 96-well plates at 2 μg/well overnight at room temperature. The unbound collagen is washed away before binding experiments. Fluorescently (NBD)-labeled immunoliposomes and control vesicles diluted in PBS containing 0.1% BSA are added into collagen wells. The incubation is for 2 hours at room temperature with mild agitation. The plate is washed in the binding buffer 3 times after binding. The amount of bound vesicles is quantified by measuring the total fluorescence signal of each well in a FlexStation (Molecular Device) with 460 nm and 580 nm as excitation and emission, respectively.

### EXAMPLE 1

### Binding of Collagen-Antibody-Conjugated Immunoliposomes is Specific for a Collagen Surface

Immunoliposomes were prepared as described above. Specific binding (measured as fluorescence) of collagen-antibody-conjugated vesicles to collagen surface as a function of nmoles of lipid was measured.

FIG. 1 is a graph of the specific binding (measured as fluorescence) of collagen-antibody-conjugated vesicles to collagen surface as a function of nmoles of lipid. Collagen-antibody-conjugated vesicles bind to collagen surface in a dose-dependent manner (col-Ab vesicle/col), but not to BSA coated surface (col-AB vesicle/BSA). The binding of un-conjugated control vesicles to collagen surface under the same conditions is shown as unconjugated vesicle/col. All vesicles contain 20% PS.

### EXAMPLE 2

### Measurement of Procoagulant Activity; Thrombin Generation Assay for the Procoagulant Activity of Vesicles

Purified human factor Xa, Va and prothrombin are purchased from Haematologic Technologies, Inc. and thrombin substrate (H-D-HHT-Ala-Arg-pNA.2AcOH) is purchased from American Diagnostica Inc. The assay is carried out in TBS, pH 7.5, with 5 mM Ca²⁺, 0.1% BSA, 1 nM of factor Xa, 5 nM of factor Va, 0.5 μM of thrombin substrate and phospholipid vesicles at indicated concentrations. The reaction is initiated by adding 0.5 μM prothrombin and OD₄₀₅ is monitored over time for kinetic assay. Thrombin activity generated during the assay is expressed as the Vₘₐₓ of the reaction.

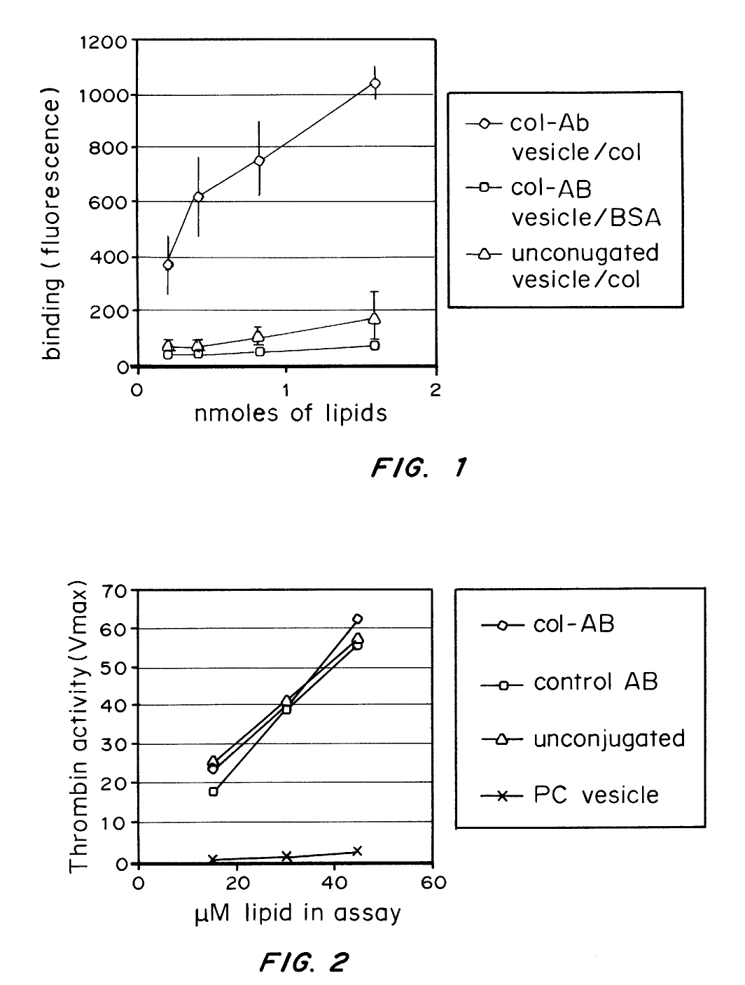

FIG. 2 is a graph of the procoagulant activity (measured as thrombin activity, Vmax) of vesicles after conjugation of anti-collagen antibody on PS-containing vesicle (as a function of micromolar lipid in assay), showing that conjugation does not affect the procoagulant activity of the vesicles. 4 vesicle preparations, collagen-antibody-conjugated vesicles with 20% phosphatidylserine (PS, col-AB), control-antibody-conjugated vesicles with 20% PS (control AB), un-conjugated vesicles with 20% PS (unconjugated) and un-conjugated phosphatidylcholine (PC) vesicles (PC vesicle) were tested in thrombin generation assay. Vesicles contain 20% porcine brain PS, 1% NBD-PC, 3% PEG-2000-DSPE (1,2-Distearoyl-sn-Glycero-3-Phospoethanolamine-N-[Methoxy(Polyethylene glycol)-2000]), 1% PEG-2000-DSPE-MAL, and 75% egg PC. In PC vesicle, 20% PS is replaced with PC. In this experiment, there are 31 antibodies per vesicle on average for antibody conjugated vesicles.

### EXAMPLE 3

### PS-Containing Vesicles Conjugated with Collagen Antibody are Procoagulant when they are Specifically Targeted to Collagen Surface

Vesicles prepared as described above are allowed to bind to collagen or a BSA coated plate. After the unbound vesicles are washed away, the thrombin generation assay is performed for each sample.

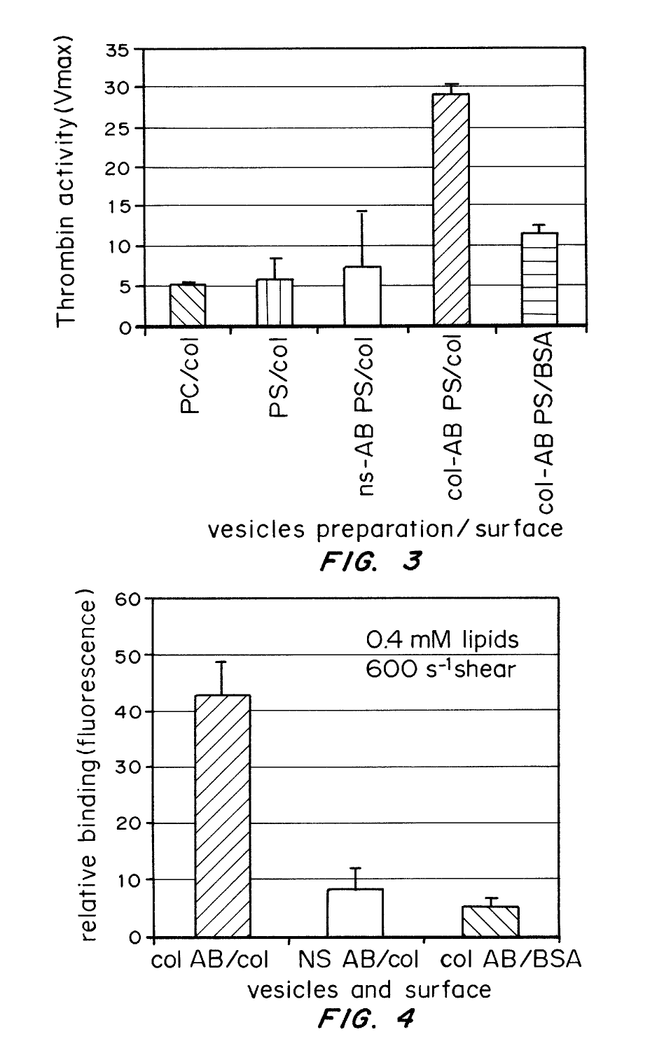

FIG. 3 is a graph of the thrombinase activity on PS-containing vesicles conjugated with collagen antibody, showing they are procoagulant when they are specifically targeted to collagen surface. The graph shows thrombin activity generated in each sample. Shown in the graph from left to right are: un-conjugated PC vesicles on collagen; un-conjugated PS vesicles on collagen; non-specific-antibody-conjugated PS vesicles on collagen; collagen-antibody-conjugated PS vesicles on collagen and collagen-antibody-conjugated PS vesicles on BSA.

### EXAMPLE 4

### Binding of Immunoliposomes on Collagen Surface Under Flow

0.2×2.0 mm flat glass capillaries (Vitrotubes™) are coated with collagen (Sigma, c4407, human placenta, type X) dialyzed in 100 mM phosphate buffer, pH 7.4, or BSA for overnight at room temperature. Lipid vesicles are diluted at 0.4 mM in TBS with 0.1% BSA. After the capillaries are washed with buffer, vesicles are perfused through the capillaries at the indicated shear rate of 600 S⁻. Capillaries are washed with 3 volumes of buffer and the amount of fluorescently-labeled vesicles bound to the collagen surface in the capillaries is examined and quantified by fluorescence microscopy.

FIG. 4 is a graph showing specific binding of collagen-antibody-conjugated vesicles to collagen surface under flow condition. Binding of each vesicle preparation is expressed as the mean fluorescence of a capture field, n=4. All vesicles contain 20% PS and the composition of vesicles is the same as described above.

### EXAMPLE 5

### Enhancement of Fibrin Formation by Collagen-Antibody-Conjugated Vesicles

ChronoLog collagen (CHRONO-PAR™, #385) is diluted in equal volume of SKF isotonic glucose solution, pH 2.7-2.9 (Kollagenreagens Horm), to make a final concentration of 0.5 mg/ml. Tissue factor (Dade Innovin, Dade Behring) is added to the collagen solution at various dilution ratios. Capillaries are coated with collagen a day ahead of the experiment at room temperature. The capillaries are briefly washed in C buffer immediately before perfusion of blood samples. Alexa-546-conjugated fibrinogen (Invitrogen) is added to blood samples at the final concentration of 30 μg/ml. Blood is collected in 2.4 μm (final) Integrilin (a GPIIbIIIa antagonist). Immediately after blood collection the blood samples are perfused through the collagen:tissue factor coated capillary at a shear rate of 300 s⁻¹. The amount of fibrin formed in capillaries is visualized by fluorescent microscope and quantified by the mean fluorescence of images captured.

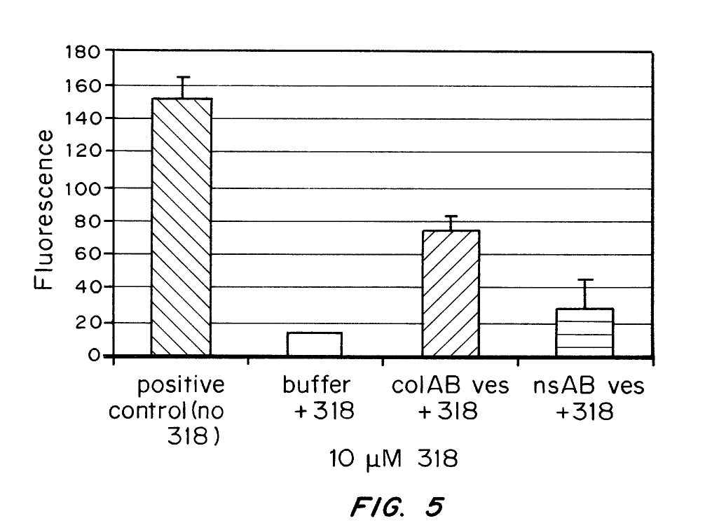

FIG. 5 is a graph of formation of fibrin by collagen-antibody-conjugated vesicles on collagen surface in whole blood when platelets are inhibited by 318, an inhibitor of platelet adhesion and activation. Fibrin formation by collagen-antibody-conjugated vesicles was enhanced in the presence of 318, demonstrating that collagen-antibody-conjugated vesicles in whole blood can bind to a collagen surface and enhance procoagulant activity, as measured by fibrin generation.

### EXAMPLE 6

### Immuno-Histochemical Staining of Mouse Femoral Artery Sections Using Immunoliposomes

Frozen mouse artery imbedded in OCT is sectioned and mounted on microscopic slides. The tissue section is fixed with cold acetone. Immuno-histochemical staining of the tissue section is carried out using Vectstain™ ABC Kit from Vector Laboratories (Burlingame, Calif.). Immunoliposomes and liposomes conjugated with a non-specific control antibody are used as primary antibodies in the procedure. Binding of antibody-conjugated immuniliposomes is visualized and quantified by colorimetric detection.

The visualized results demonstrate that the collagen-antibody-conjugated immunoliposomes bind to mouse femoral artery sections containing exposed collagen.

Modifications and variations of the present invention will be obvious to those skilled in the art from the foregoing detailed description. Such modifications and variations are encompassed by the following claims. References cited herein are specifically incorporated by reference.

We claim:

1. A platelet substitute comprising
large unilamellar lipid vesicles comprising 1-phosphatidylserine in combination with other lipids, and
one or more proteins selected from the group consisting of proteins having binding affinity for a component of the blood vessel wall that becomes exposed upon vessel injury.

2. The platelet substitute of claim 1 further comprising at least one molecule of a phospholipid scramblase per vesicle.

3. The platelet substitute of claim 1 comprising a protein targeting to and adhering to subendothelium bound to the outer surface of the lipid vesicle.

4. The platelet substitute of claim 1 comprising an amount of 1-phosphatidylserine (PS) effective to provide procoagulant function.

5. The platelet substitute of claim 4 comprising 1-phosphatidylserine in combination with other lipids selected from the group consisting of cholesterol or other sterol, phosphatidylcholine (PC), sphingomylin (SM), and phosphatidylethanolamine (PE).

6. The platelet substitute of claim 1 comprising asymmetric lipid vesicles selectively incorporating a procoagulant phospholipid within the inner leaflet of the vesicle membrane, wherein the outer leaflet of the vesicle membrane consists of phosphatidylcholine or another neutral phospholipid.

7. The platelet substitute of claim 1 comprising
a protein targeting to and adhering to subendothelium and a phospholipid scramblase protein,
wherein the targeting protein and scramblase protein are incorporated into or covalently bound to the membrane of the lipid vesicle.

8. The platelet substitute of claim 7 wherein the targeting protein and the scramblase form a single chimeric protein construct comprising the protein targeting to and adhering to the subendothelium covalently linked through a transmembrane amphipathic helix to a phospholipid scramblase is incorporated into the membrane of the lipid vesicle,
Wherein the subendothelium-targeting domain of the chimeric protein is exposed on the external surface of the lipid vesicle and the phospholipid scramblase domain of the chimeric protein is located on the internal surface of the lipid vesicle.

9. The platelet substitute of claim 5 wherein phospholipid 1-phosphatidylserine is concentrated in the inner leaflet of the vesicle membrane and neutral lipids are concentrated in the outer leaflet of the vesicle membrane.

10. The platelet substitute of claim 3 wherein the targeting protein comprises the extracellular domain of human platelet glycoprotein GPIa-IIa, which is covalently linked to phospholipid scramblase 1 (PLSCR1) through a transmembrane domain that is capable of facilitating leakage of calcium ion across the vesicle membrane.

11. The platelet substitute of claim 3 wherein the targeting protein is a receptor or antibody for collagen, human platelet glycoprotein Ib or von Willebrands Factor.

12. The platelet substitute of claim 2 wherein the phospholipid scramblase is selected from the group of human phospholipid scramblase 1-4, consisting of human phospholipid scramblase 1 (PLSCR1), PLSCR2, PLSCR3, and PLSCR4.

13. The platelet substitute of claim 1 wherein the targeting protein is covalently coupled directly to the phospholipid head group of the lipid vesicle, wherein the phospholipid is phosphatidylethanolamine comprising lipid-bilayer-perturbing fatty acid chains attached at the SN1 or SN2 positions of the glycerol backbone of the phospholipid.

14. The platelete substitute of claim 1 comprising a pegylated phospholipids.

15. The platelet substitute of claim 1 comprising fluorescently-labeled phospholipid, either in the membrane bilayers or entrapped inside the immunoliposome.

16. The platelet substitute of claim 1 wherein the platelet substitute comprises antibodies targeted to a collagen surface.

17. The platelet substitute of claim 1 comprising antibody conjugated liposomes providing procoagulant activities in human blood in which platelet function is completely inhibited.

18. The platelet substitute of claim 1 lyophilized or dried in a sterile dosage unit container.

19. The platelet substitute of claim 1 stored at −20° C. or 4° C.

20. The platelet substitute of claim 1 suspended in a pharmaceutically acceptable solution for administration to a patient in need thereof.

21. A method for promoting coagulation comprising administering an effective amount of the platelet substitute of claim 1 to an individual in need thereof.

22. The method of claim 21 comprising providing an effective amount of the platelet substitute to a patient before, during or after surgery.

23. The method of claim 22 comprising administering the platelet substitute directly to a wound.

24. A method of making a platelet substitute comprising providing large unilamellar lipid vesicles comprising a procoagulant amount of 1-phosphatidylserine in combination with other lipids, and inserting into the vesicles or covalently binding to the phospholipids one or more proteins selected from the group consisting of proteins having binding affinity for a component of the blood vessel wall that becomes exposed upon vessel injury.

25. The method of claim 24 comprising selectively incorporating a procoagulant phospholipid within the inner leaflet of an asymmetic vesicle membrane, wherein the outer leaflet of the vesicle membrane consists of phosphatidylcholine or another neutral phospholipid.

26. The method of claim 24 comprising providing in the vesicles a protein targeting to and adhering to subendothelium and a phospholipid scramblase protein, wherein the targeting protein and scramblase protein are incorporated into or covalently bound to the membrane of the lipid vesicle.

27. The method of claim 26 wherein the targeting protein and the scramblase form a single chimeric protein construct comprising the protein targeting to and adhering to the subendothelium covalently linked through a transmembrane amphipathic helix to a phospholipid scramblase is incorporated into the membrane of the lipid vesicle,
wherein the subendothelium-targeting domain of the chimeric protein is exposed on the external surface of the lipid vesicle and the phospholipid scramblase domain of the chimeric protein is located on the internal surface of the lipid vesicle.

28. The method of claim 24 wherein the targeting protein is covalently coupled directly to the phospholipid head group of the lipid vesicle, wherein the phospholipid is phosphatidylethanolamine comprising lipid-bilayer-perturbing fatty acid chains attached at the SN1 or SN2 positions of the glycerol backbone of the phospholipid.
